(19) United States
(12) Patent Application Publication
(10) Pub. No.: US 2016/0103307 A1
(43) Pub. Date: Apr. 14, 2016

(54) METHOD AND SYSTEM FOR STOKES INTERFERENCE STIMULATED FLUORESCENT SCATTERING FOR IN-VIVO IMAGING

(71) Applicant: Robert David Frankel, Rochester, NY (US)

(72) Inventor: Robert David Frankel, Rochester, NY (US)

(21) Appl. No.: 14/881,701

(22) Filed: Oct. 13, 2015

Related U.S. Application Data
(60) Provisional application No. 62/063,017, filed on Oct. 13, 2014.

(51) Int. Cl. G02B 21/00; G02F 1/11; G02B 26/10; A61B 5/00

(52) U.S. Cl. CPC G02B 21/0076; A61B 5/0071; G02B 21/0048; G02B 21/0056; G02B 21/0028; G02B 21/0032; G02F 1/11; G02B 26/101

(57) ABSTRACT

A microscopy system includes a first laser emitting a first laser pulse, the first laser pulse being a pump beam; a second laser emitting a second laser pulse, the second laser pulse being spectrally isolated for generating a probe beam and a donut beam; an optical device for combining the pump beam, the probe beam and the donut beam into a combined laser pulse, the probe beam and donut beam having a phase difference that causes a reduction of a focal volume of the combined laser pulse; a galvanometer scanning system for delivering the combined laser pulse to a focal spot in a focal plane, wherein the reduction of the focal volume of the combined laser pulse initiates a stimulated emission of a targeted molecule, the stimulated emission having dipole-like backscatter; and a sensor for enabling imaging of the dipole-like backscatter.

### BACKGROUND

The subject matter described herein relates to method and system Stokes Interference Stimulated Fluorescent Scattering for in-vivo imaging.

There is significant interest in providing label-free auto-fluorescence imaging for use in in-vivo imaging for research, endoscopy, dermatology and intra-surgical definition of clear margins during removal of malignant tissues. Certain chromophores, e.g. beta-carotene, oxy-hemoglobin, deoxy-hemoglobin, melanin, cytochromes and certain states of the metabolic cofactors NADH and FAD, absorb light but do not fluoresce. This is because their spontaneous emission is dominated by their fast non-radiative decay (which can be four orders of magnitude faster than their rate of spontaneous emission) from an excited state. The distribution of this class of molecules in cells has been measured with Stimulated Fluorescent Emission (SFE) techniques. SFE images have been obtained from hemoglobin in capillaries, and diffusion of drugs, both of which were not previously possible because of poor or non-existent fluorescent emission.

Standard fluorescent emission (SFE) is an incoherent process where emission occurs in all directions; that is into and out of the tissue where the molecule is located. Unfortunately in SFE imaging, stimulated emitted light is emitted in the forward direction deeper into the tissue. Forward scattered stimulated emission means that multiple scattering events are required to redirect emission backward to create a signal outside of the tissue. This multiple scattering reduces the depth and resolution of tissue imaging.

### SUMMARY

The disclosed technology relates to a system and a method for Stimulated Fluorescent Scattering (SFE) for probing a fluorescent signature of a sample. A method called Stokes Interference Stimulated Fluorescent Scattering (SISFE) is disclosed that adds an annular beam with intense lobes above and below focus created by a π phase plate to reduce a microscope's 3-D focal spot to sub-wavelength dimensions. A sub-wavelength focal volume emits a dipole pattern of SFE with forward and backscatter lobes, enabling high resolution single backscatter imaging from deep within tissues with resolution beyond the diffraction limit. The disclosed SISFE technology can be used to measure the concentrations of both the fluorescent and non-fluorescent states of the enzyme cofactors NADH and FAD to map the metabolic state of the tissue under study as well as the mapping of many chromophores that are not fluorescent.

In one implementation, a microscopy system comprises: a first laser emitting a first laser pulse, the first laser pulse being a pump beam; a second laser emitting a second laser pulse, the second laser pulse being spectrally isolated for generating a probe beam and a donut beam; an optical device for combining the pump beam, the probe beam and the donut beam into a combined laser pulse, the probe beam and donut beam having a phase difference that causes a reduction of a focal volume of the combined laser pulse; a galvanometer scanning system for delivering the combined laser pulse to a focal spot in a focal plane, wherein the reduction of the focal volume of the combined laser pulse initiates a stimulated emission of a targeted molecule, the stimulated emission having dipole-like backscatter; and a sensor for enabling imaging of the dipole-like backscatter.

In some implementations, the first laser pulse can have a Gaussian beam profile and a sub-picosecond duration.

In some implementations, the microscopy system can further comprise: an acousto-optic modulator for modulating the pump beam on and off. In some implementations, the sensor can generate an imaging signal corresponding to a gain in intensity of the probe beam computed as the difference between the combined laser pulse with the pump beam on and the combined laser pulse with the pump beam off.

In some implementations, the microscopy system can further comprise: a Virtual Imaging Phase Array (VIPA) for spectrally isolating the probe beam and the donut beam from the second laser pulse.

In some implementations, the microscopy system can further comprise: a π phase plate for forming the donut beam.

In some implementations, the probe beam and the donut beam can be sub-picosecond laser pulses of a Stokes module, the probe beam and the donut beam are shifted from a wavelength of the pump laser and directly stimulate emission into a ground state electronic manifold.

In some implementations, the microscopy system can further comprise: an optical delay for adjusting pathlengths of the probe beam and the donut beam.

In some implementations, the combined laser pulses can be delivered in a diffraction limited spot in a focal plane of a high numerical aperture (NA) microscope objective. In some implementations, the combined laser pulses can be used to excite an electron into an electronic excited state that emits stimulated emission from its lowest energy excited state level. In some implementations, the galvanometer scanning system can move the focal spot in an X,Y plane. In some implementations, the probe beam and the donut beam can be emitted so as to arrive at the focal spot after the pump beam. In some implementations, the probe beam and the donut beam can initiate stimulated emission from an excited state of the targeted molecule.

Advantages of SISFE include, for example, label-free imaging of a tissue's microvascular structure, based on endogenous contrast from non-fluorescent hemoglobin, which has been imaged by SFE in the forward scatter direction. Metabolic imaging of the relative amounts of reduced NADH and FAD and the microenvironment of these metabolic electron carriers can be used to noninvasively monitor changes in metabolism, which is one of the hallmarks of carcinogenesis, e.g., when cofactors are bound to metabolic enzymes, NADH fluorescence quantum yield increases, while FAD quantum yield decreases, which causes variation in the measured fluorescence intensities.

SISFE techniques can also measure both bound and unbound cofactor concentrations and spatially resolve both molecular states. Another aspect of the disclosed technology described is the application of SISFE techniques to determine the metabolic state of cells, by measuring the fluorescent and non-fluorescent concentration of these metabolic co-factors.

In another implementation, in order to reduce the backscattered absorption, 4-photon stimulated emission can be introduced to increase the wavelength of the backscattered radiation to enable deeper SISFE penetration and to enable deeper multiple scatter backscatter collection.

### BRIEF DESCRIPTION OF THE DRAWINGS

FIG. 1 is a block diagram of an example of a system used with the disclosed technology;

FIG. 2 is a block diagram of another example of a system used with the disclosed technology;

FIG. 3 is a block diagram of another example of a system used with the disclosed technology;

FIG. 4a is a graphical depiction of donut intensity;

FIG. 4b shows a π phase plate;

FIGS. 5a-c are graphical depictions of schematic SISFE energy and laser parameters;

FIGS. 6a-c are graphical depictions of SISFE Point Spread Functions in the axial direction.

FIG. 7a-b are graphical depictions of backscatter transverse focal spot size, switched performance and system;

FIG. 8 is a graphical depiction of pulse timing of Stokes pulses relative to variable fluorescent lifetimes for free and bound NADH;

FIG. 9 is an illustration of directions of stimulated emission lobes in SFE and SISFE;

FIG. 10a-c are graphical depictions of energetic transitions of 2-photon, 3-photon and 4 photon stimulated emission in SFS and SISFS; and

FIG. 11 is a pictorial diagram of an example of a microscope aperture area used with the disclosed technology.

### DETAILED DESCRIPTION

The subject matter described herein relates to method and system for Stokes Interference Stimulated Fluorescent Scattering for in-vivo imaging. Specifically, the subject matter described herein relates to fluorescence microscopy and more particularly to in-vivo stimulated fluorescence to image the backscatter stimulated fluorescent emission of specific molecules. The subject matter described herein can be applied, for example, to measuring metabolism of cells in-vivo as well as signals from proteins, DNA and RNA.

Standard fluorescent emission (SFE) is an incoherent process where emission occurs in all directions. Unfortunately in SFE imaging stimulated emitted light is emitted in the forward direction deeper into the tissue. Forward scattered stimulated emission means that multiple scattering events are required to redirect emission backward to create a signal outside of the tissue. This multiple scattering reduces the depth and resolution of tissue imaging.

The subject matter described herein overcomes the problem of forward scattering. That is, an approach to SFE imaging is disclosed called Stokes Interference standard fluorescent emission (SISFE). SISFE imaging narrows a focal spot of a beam in 3 dimensions to much less than the imaging wavelength, creating dipole-like emission; thus enabling direct backscatter dipole in-vivo stimulated fluorescence imaging. In other words, the disclosed technology provides a system for direct backscatter coherent stimulated fluorescence imaging via the technique of focal spot reduction to enable dipole-like backscatter imaging.

Three laser beams are used in Stokes Interference SFE (SISFE) imaging. As in SFE imaging, there is a pump and a probe beam to cause stimulated emission. However, an additional third beam, the donut, which is focused to an annulus, is used. The donut beam narrows the region of probe beam stimulated emission.

In order to reduce the probe field strength the probe and donut can destructively interfere with each other. Thus both the probe and donut are composed of two sub-picosecond spectrally separated pulses with center wavelengths less than 10 nm apart, and of opposite phase near the temporal center of the pulses. The pump field is composed of a broadband sub-picosecond Gaussian pulse. The pump pulse precedes the probe pulse by at least several hundred femtoseconds to allow the decay of an excited state vibrational level into the lowest excitation level in the excited state manifold via a Kasha decay process.

At focus in SISFE, the radial increasing intensity of the donut reduces the amplitude of the molecular polarization at the pump-probe difference frequency, and shifts the phase of the molecular polarization out of phase alignment for probe beam stimulated fluorescent gain. Increasing the donut intensity relative to the probe drives the probe gain suppression closer to the optic axis of the system, thus narrowing the diameter of the probe stimulated emission spot. The donut beam also causes narrowing of the emission spot along the optic axis of the laser beams. This is because, in SISFE, the donut beam is formed by a π phase plate which produces an annular beam at focus and intense lobes above and below the plane of focus, which reduces the focal spot size along the optic axis by 3-4 times the confocal axial diameter.

In in-vivo imaging, the reduction of the emission spot to less than 80% of the probe wavelength in all three spatial dimensions alters the stimulated emission field distribution at focus. These focal spots size the sample emission characteristics by changing a volume forward stimulated emission, as in a laser, to bi-directional forward and backward dipole stimulated emission. Half of the stimulated emission goes directly back out of the tissue from the single stimulated event. Multiple photon scattering events are not required as they are in backscatter SFE imaging. Therefore, there is significantly less signal loss in signal collection, enabling fast and deeper signal collection from the tissue.

There is another change in signal characteristics with backscatter detection. In the forward direction, the stimulated emission interferes directly with the probe beam. To detect a significant signal, a large number of photons can to be used. However, in backscatter detection the stimulated emission interferes with the normal tissue backscatter caused by small changes in the refractive index of the tissue. This is many orders of magnitude weaker than the forward scattering beam. Therefore, a statistically significant signal is achieved with fewer photons.

In metabolic imaging, various delays between the pump and Stokes pulses can be used. This enables the measurement of the concentration of membrane bound and un-bound NADH and FAD which have different excited state lifetimes.

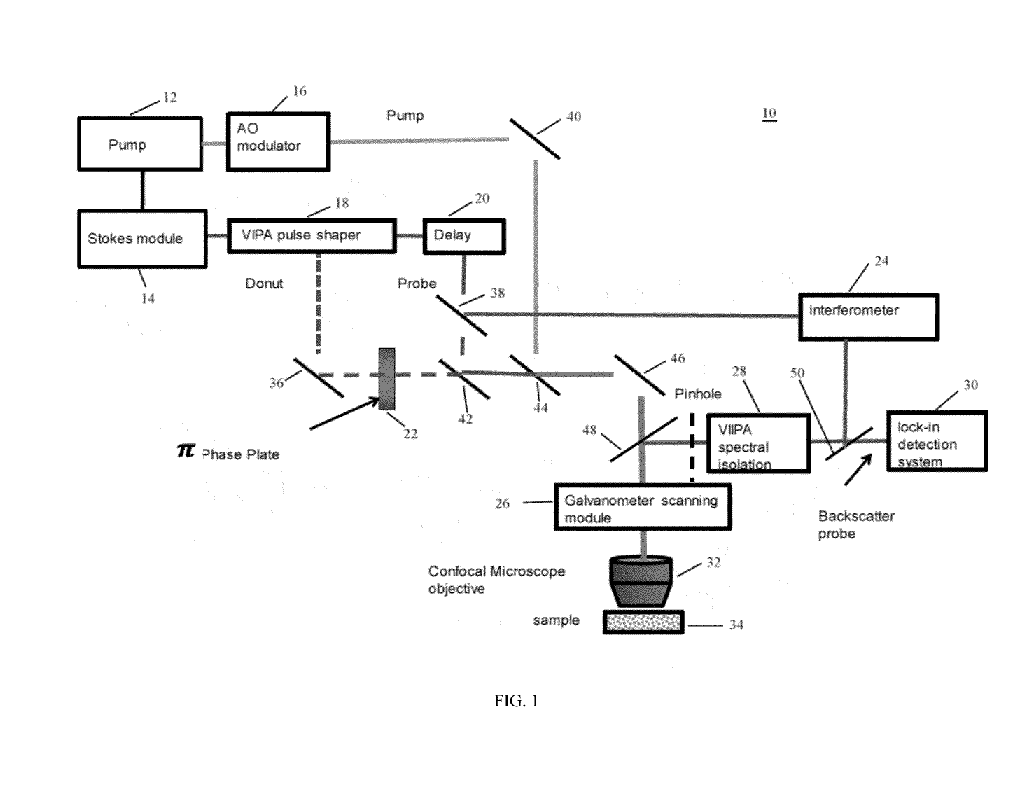

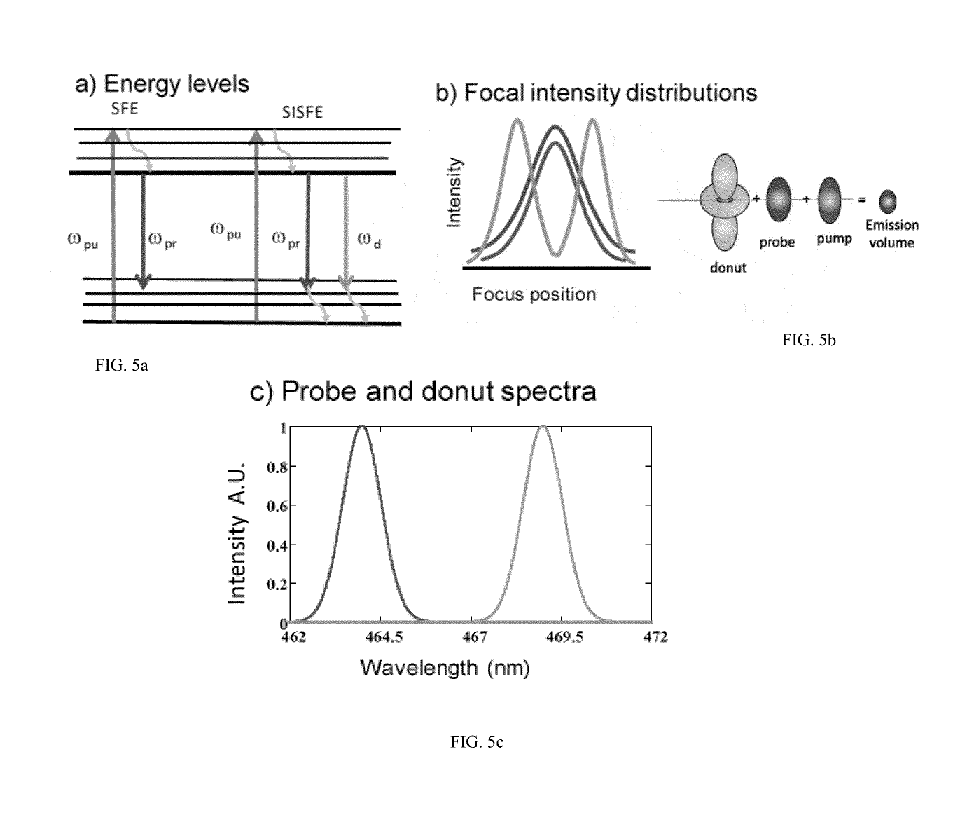

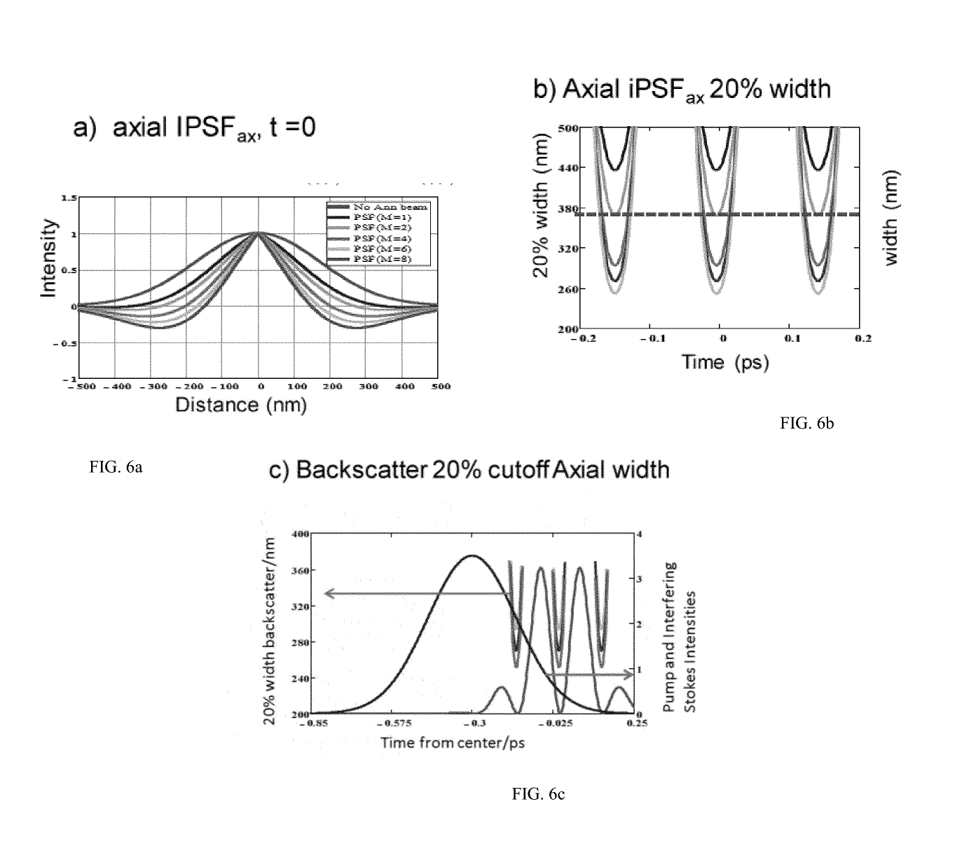

FIG. 1 schematically shows a system 10 for scanning Stokes Interference Stimulated Emission (SISFE) microscope. In FIG. 1, a microscopy system 10 focuses a pump beam from a pump laser 12 emitting laser pulses with a Gaussian beam profile and sub-picosecond duration in a diffraction limited spot in the focal plane of a high numerical aperture (NA) microscope objective 34. A galvanometer scanning system 26 moves the spot around in the X,Y plane. A sample 34 to be investigated is located in or near the focal plane. The laser pulse is used to excite an electron of the sample 34 into the electronic excited state that will emit stimulated emission from its lowest energy excited state level. Sub-picosecond laser pulses from an additional laser in a Stokes module 14 creates two laser pulses, the probe and the donut, both of which are shifted from the wavelength of the pump laser, and directly stimulate emission into the ground state electronic manifold. These pulses are referred to as “Stokes” pulses and are used to initiate stimulated emission from the excited states of the sample 34. The sub-picosecond Stokes laser pulses are emitted so as to arrive at the focal spot after the pump beam, as shown in FIG. 6c. This is because the pump excites a higher electronic excited level in the molecule under study, and the electron has to drop into the lowest level in the excited state before stimulated emission via a Kasha decay process, as shown in FIG. 5a. This process can take, e.g., 0.01 ps-2 ps or 0.1 ps-1 ps.

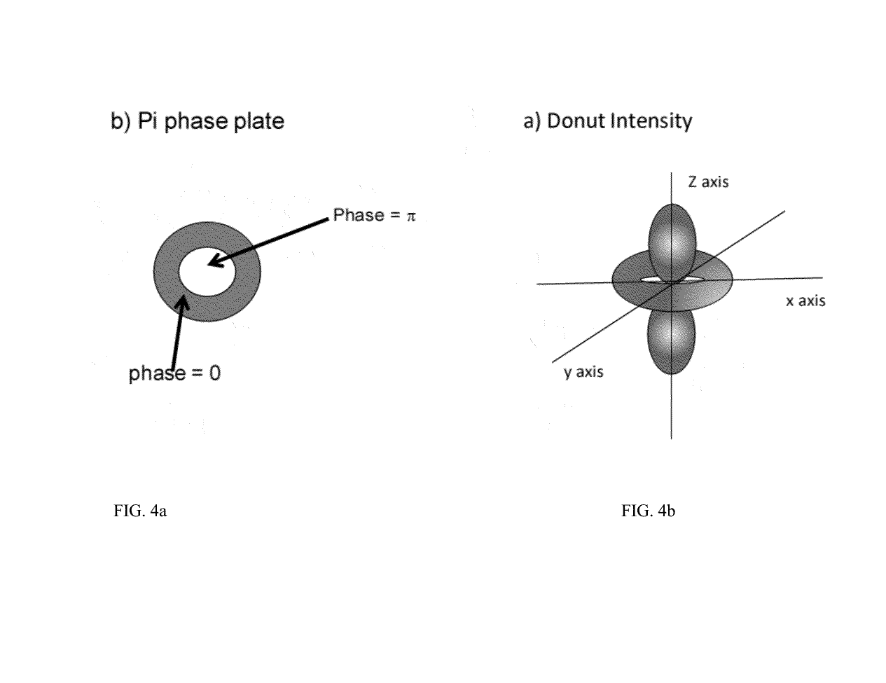

The pathlengths of the probe and donut are adjusted by placing an optical delay line 20 with movable mirrors 38, 40, 42, 44, 46, 48 in the laser beams. The pump beam is modulated on and off by an acousto-optic modulator 16. The donut beam is formed by the π phase plate 22, shown in FIG. 1 and FIG. 4. The pump laser beam and the two Stokes laser beams are combined using conventional optics, e.g. reflective and dichroic mirrors, beam splitters and lenses.

The two Stokes beams have specific characteristic wavefronts that enable imaging beyond the diffraction limit. In FIG. 5b, the focused Stokes donut laser beam has an annular intensity profile shape, for example, in the form of a torus of intensity with substantially zero in intensity at the center of the focal point of the pump beam in the focal plane, as well as high intensity lobes above and below focus as shown in FIG. 5 (b). Also the donut inner surface of the torus can be modeled to have the shape of a sine wave of revolution around the center of the microscope focus.

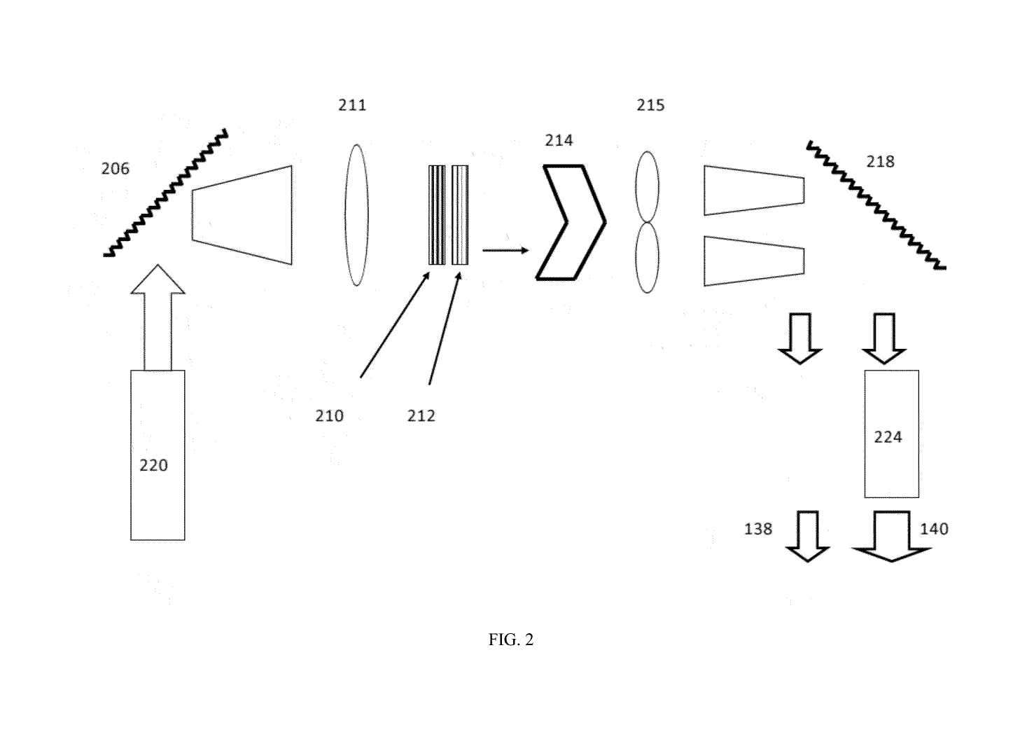

In the embodiment shown, the donut and probe beams are formed from the output of a single laser that is put through a grating or Virtual Imaging Phase Array (VIPA) 218 as shown in FIG. 2. The VIPA 218 spectrally isolates the probe and donut beams. In another implementation, the Stokes beams can also be formed from coupled laser cavities, or from a laser cavity operating on orthogonal modes.

It is important that the donut and probe laser beams have a controlled phase and amplitude relationships between each other. In addition, it is important that they are close together in wavelength as their central wavelength can be separated by less than the homogenous bandwidth of the stimulated emission levels. That is, the central wavelengths can be separated by less than 50 nanometers or less.

FIG. 2 shows a method of generating two stimulated emission pulses that are close together in wavelength and have a controlled phase and amplitude difference. These two beams can be generated from a single picosecond seed laser, e.g. a Ti: Sapphire solid state laser 220. The output beam 202 of laser 220 is spectrally dispersed by diffraction grating 206. The spectrally dispersed beam is collimated by lens 211. The phase of the frequency components of the beams can be changed by a first liquid crystal array 210 and the polarization can be altered by a second liquid crystal array 211. This is similar to the arrangement used in picosecond laser quantum phase control laser chemistry experiments. The spectral components of each of the two Stokes pulses can then be spatially separated by tilted plates 214. The two beams are then refocused by lens 215 and the spectral components of each beam are then recombined by grating 218. Laser amplifier 224 can then donut Stokes beam 140 which can be 4-15 times more intense than pump laser beam 138. The wavelength separation of the two Stokes lasers can be defined by the bandwidth of the electronic levels being probed.

As shown in FIG. 1, the pump laser 12 can be modulated by an acousto-optic modulator 16 and the probe beam can be recorded in a lock-in detection system 30 that operates at 1 megahertz or above. The difference in the backscatter with the pump on and with the pump off determines the probe beam gain. A dispersion module 28 can be used in front of the lock-in amplifier to isolate the probe backscatter from the donut backscatter.

A portion of the probe before being incident on the sample is picked off and directed into an interferometer 24 that interferes with the incident probe of the backscattered probe. This interferometer 24 is used to measure the position and phase of the centroid of the refractive index gradient induced backscatter relative to the plane of best focus when the probe beam is turned off. Alternatively other backscatter methods can be used to measure this position, e.g. placing a reflecting plate in the converging focused beam, but out of the focal spot that serves as an in-line interferometric reference.

The backscattered stimulated emission probe signal can be isolated by suitable spectrometers. The photons produced from donut and probe emission can be separated. The imaging signal corresponds to the gain in intensity of the probe beam, computed as the difference in the emitter pumped minus the emitter un-pumped beam. The gain in the donut Stokes beam is not the image signal.

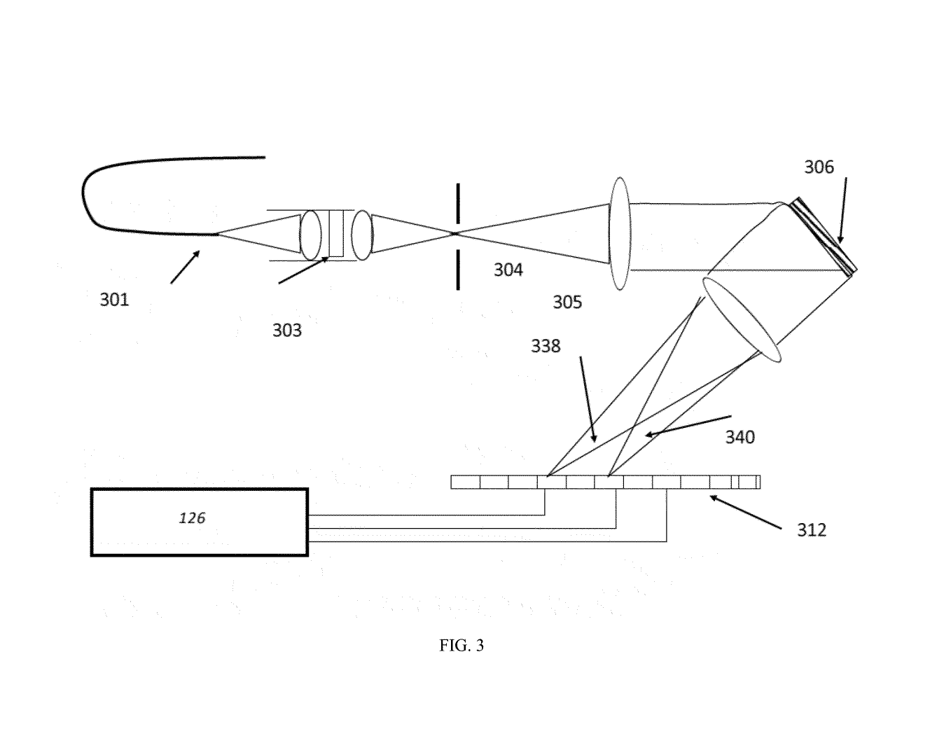

FIG. 3 shows a photon detection spectroscopy system used to collect signals with SISFE microscopy. The spectrometers can be implemented as a conventional grating or Virtual Image Phase Array (VIPA) spectrometer isolation unit. The grating spectrometer, in this example VIPA isolation unit in of FIG. 1, receives light through an optical fiber 301. The optical fiber 301 can be a multimode fiber or a single mode fiber. Multimode fibers can have cores larger than 50 microns or more and hence will collect more light. Alternatively, free space optics can be used instead of an optical fiber. The light is detected in the backward scattered direction by the “epi” light lock-in system, using a VIPA spectrometer constructed in a similar manner. Epi scattering can be caused by multiple scattering beyond the focus of the laser, which can occur in thick samples, or by the dipole backscattered radiation component. The pinhole prior to the isolation detector isolates the dipole signal for the multi-scattered noise. The spectrometer can be positioned remotely or integrated into the microscope system. The light detection system of the spectrometer can include silicon avalanche photodiodes 312, fast photodiodes, or photomultiplier tubes.

FIG. 3 shows a signal collection optical fiber 301. The output of the fiber is collimated and passes through pump optical filter 303 to remove the excitation pump light. This filter can be a multilayer filter to remove excitation light. The light is then focused through the slit entrance 304 to a grating, or VIPA spectrometer, the grating 306 spectrally disperses and separates the stimulated emission enhanced central probe beam 338 and probe 340 Stokes beam. These beams are then focused by lens 304 on to an array of detectors 312. The controller 126 controls signal acquisition and constructs the image. The signal for each image pixel is the difference between the central Stokes beam with the pump beam turned on to excite the sample and with the pump beam turned off. This is the “gain” in the stimulated emission from the probe beam 338.

FIG. 5a is an energy diagram of SFE and SISFE. The pump ωₚᵤ (blue) and probe ωₚᵣ (red) beams are used in SFE to stimulate photon emission into the probe beam and to populate vibrational level ωᵥ. In SISFE imaging the donut beam ω_{d} (green) is added. Both the probe and donut stimulate fluorescent emission into a molecular ground state manifold. The energy level diagram and focal spot spatial distributions of SFE and SISFE imaging are outlined in FIG. 5a. In SFE, two laser beams at the pump frequency, ωₚᵤ, and probe frequency, ωₚᵣ, (sometimes called the Stokes field) are coincident on a sample. The Stokes fields have appropriate energy to drive stimulated emission into an excited state in the ground state manifold as shown in FIG. 5a.

FIG. 5b is the focal distribution of energy in focal spots of the pump, probe and donut beams. The pump and probe have Gaussian distributions. The donut is focused to an annulus in the transverse plane with bright intensity nodes above and below focus. The Stokes components are π radians out of phase at the center of the pulses. As shown in FIG. 5b the donut beam has an annular intensity distribution with high intensity lobes above and below focus, while the pump and probe have Gaussian intensity distributions. The Stokes component pulse separation is narrow enough to cause stimulated emission in a single fluorescent transition.

FIG. 5c is the Stokes components that are separated in the detection system. In this example the probe wavelength λₚᵣ=464.0 nm and the donut wavelength is λ_{d}=469 nm. The pulses have a Gaussian ½ width of 0.3 ps.

The two Stokes components are sub-picosecond spectrally separated pulses, with center wavelengths as shown in FIG. 5c for the system parameters exemplified here. The probe and donut can have non-overlapping spectra because the probe is isolated as the imaging signal.

The Stokes components are delayed from the temporal center of the pump pulse as represented in FIG. 6c which shows the envelope of interfering Stokes components of equal intensity, as well as, the pump envelope.

The phase difference of the Stokes components determines the shape and size of the focal volume of the emitters. When the Stokes components have a phase relationship in the range of less than π±π/2 radians, the fields of the Stokes components can interfere destructively and the intensity of the Stokes field can be reduced. At focus, the radial increasing intensity of the interfering donut reduces the Stokes amplitude and changes the intensity and phase of the molecular polarization at the probe frequency, reducing the focal volume of probe gain. Increasing the donut intensity relative to the probe drives the probe gain suppression closer to the optic axis of the system narrowing the diameter of the probe stimulated emission spot.

The phase difference of the Stokes components, Φ(t) changes as a function of time throughout the pump pulse according to Eq. (1). Here c is the speed of light, λₚᵣ is the probe wavelength and λ_{d} is the donut wavelength. The further apart the Stokes components are in wavelength the shorter is the time period when they interfere.

$$Φ ( t ) = π + 2 π tc \cdot \frac{( {λ}_{d} - {λ}_{pr} )}{{λ}_{d} \cdot {λ}_{pr}} \qquad ( 1 )$$

The reduction of focal volume that occurs when the Stokes components are out of phase acts as a gate for the induction of backscatter. Efficient backscatter will occur only when the phase difference and the intensity of the donut beam is high enough to initiate dipole-like emission. That is, the focal spot size is reduced to less than 80% of the wavelength in all 3 dimensions. This turns volume forward stimulated emission into dipole-like backward and forward emission. Thus dipole scattering only occurs during less than 50% of the Stokes beat period.

Small scatterers have dipole emission patterns regardless of focal spot size. However, without a focal volume reduction forward scattered stimulated fluorescent emission occurs from the cellular mitochondria, capillaries, organelle structures, and protein filaments at random angles.

In confocal microscopes the axial focal diameter is 1-3× or 2.0-2.5× the transverse diameter. In the visible, for a 1.2 NA water objective, in order to obtain volume stimulated Raman backscatter the axial focal spot diameter can be reduced by >3×. The transverse dimension can be reduced by ˜1.5× the confocal diameter.

In a SISFE system, focal spot volume reduction is accomplished by the use a π phase plate to generate the donut beam. In addition to providing an annular intensity distribution in the plane of focus, these phase plates provide higher intensity lobes above and below the Gaussian focus of the pump and probe beams. The peak intensities of the axial lobes can be designed to be 3-4 times more intense than the transverse annular peak intensity.

In the forward direction the stimulated Raman signal interferes with the probe beam which acts as a local oscillator. In the backscattered direction the stimulated signal can interfere with the much weaker index of refraction gradient dependent backscattered photons. The phase relationship of the gradient index backscatter to the dipole SISFE signal can be variable from pixel to pixel.

In SISFE, the intensity of the pump beam is modulated at a high frequency f (>1 MHz), whereas the probe beam is unmodulated. This is because in the backscattered direction the tissue index gradient backscatter local oscillator field will be small and the dominate signal can be the non-heterodyned probe stimulated emission intensity.

In a SFE system the forward scattered stimulated electronic gain is small and measured with a lock-in amplifier system. In SISFE, the stimulated gain is an even smaller perturbation on the incident probe, in part due to the smaller excitation focal spot size. However, in the backscattered direction the stimulated signal is a larger fraction of the low intensity index gradient backscattered local oscillator beam, enabling shot noise limited backscatter gain measurement with fewer photons than forward scattered SISFE.

### SISFE THEORY

The absorption cross section, σ_{abs} for optical radiation for a single chromophore at room temperature is about 10⁻¹⁶ cm². In a tightly focused laser beam with a beam waist, S (˜10⁻⁹ cm²) the integrated intensity attenuation of the excitation pump beam ΔIₚᵤ/Iₚᵤ is proportional to the ratio between σ_{abs} and S:

ΔIₚᵤ/Iₚᵤ=−N₀σ_{abs}/S (2)

Here Nₒ is the number of molecules in the ground state. For a single chromophore, ΔI_{E}/I_{E} is of the order of 10⁻⁷. Such attenuation cannot be detected by conventional absorption microscopy. As the stimulated emission cross section σₛₜᵢₘ is comparable to the absorption cross section, the change in intensity of a stimulated probe beam is:

ΔIₚᵣ/Iₚᵣ=−Nₛσₛₜᵢₘ/S (3)

Here N₂ is the small number of molecules transiently probed by the stimulating beam. For a single chromophore ΔIₚᵣ/Iₚᵣ=10⁻⁷. Using a lock-in amplifier technique at >1 MHz sampling rate, small number of chromophores have been measured by stimulated emission.

Under a non-saturating condition of the four-level system (FIG. 5a). N₂ in equation (3) originates from a linear excitation: N₂ αN₀Iₚᵤσ_{abs}/S. This relation, together with equation (2), indicates that the final signal ΔIₚᵣ is linearly dependent on both and

ΔIₚᵣ∝N₀IₚᵤIₚᵣ(σ_{abs}/S)/(σₛₜᵢₘ/S) (4)

Stimulated gain can also be analyzed within the framework of linear induced polarization for propagating plane waves. In forward scattered SFE, the induced polarization, Pₚᵣ, is generated at the probe frequency, where and PₚᵣαX(Ω)EₚᵣIₚᵤ, where X(Ω) is the susceptibility of the medium, Eₚᵣ is the probe electric, field and Iₚᵤ is the pump intensity.

The pump excitation pulse populates a higher level excited state that decays to the lowest energy level in the excited state manifold via a kasha decay process on a sub-picosecond decay time. In certain applications of SISFE, a further delay in the Stokes pulses can be used to isolate populations of the fluorescent and non-fluorescent states of certain molecules of different lifetimes e.g. membrane bound and free NADH and FAD. Thus the propagating polarization also depend on the time delay between the pump and Stokes pulses, as the population in the excited state decay is dependent on the excited state lifetime, prior to stimulated emission. For large focal volumes Pₚᵣ propagate in the forward direction and interfere with the incident pump and probe fields with the corresponding phases. For forward scattered stimulated gain Pₚᵣ interferes constructively with Eₚᵣ and results in intensity gain, Gₚᵣ, which scales as the product of the probe and pump intensities Iₚᵣ and Iₚᵤ.

The equations for Stokes polarization, Pₛₜ(ω), in SISFE are shown in Eq. (5). In SISFE the Stokes field is Eₛₜ=Eₚᵣ+E_{d} the sum of the probe and donut fields. Both difference frequencies coincide with the molecular susceptibility response function X(ω).

Pₛₜ(ω)∝Eₚᵣ(ω)·Iₚᵤ(ω)·e^{-Δtτ}·χ(ωₚᵣ)+E_{d}(ω)·Iₚᵤ(ω)·e^{-Δtτ}·χ(ω_{d}) (5)

Here Δt is the delay between the peak of the pump pulse and the peak of the Stokes pulses, τ is the excited state decay constant and e^{-Δtτ} the excited state decay prior to the arrival of the stimulated emission pulses. The two product terms in Eq. (2) are designated as the probe polarization Pₚᵣ and the donut polarization P_{d}. Therefore, the induced polarization at focus is, Pₛᵢ=Pₚᵣ+P_{d}.

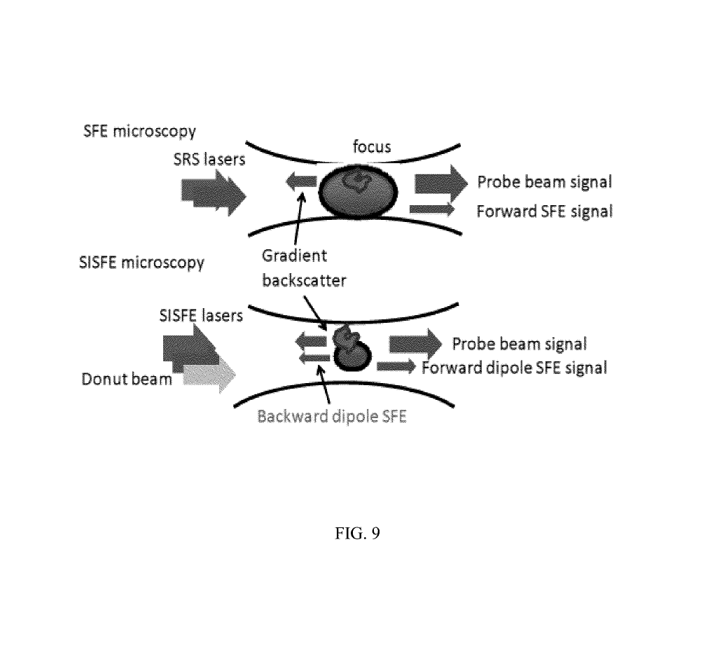

The SISFE microscopy system takes into account the π radian Gouy phase shift in the stimulated emission excitation fields through focus of a high NA microscope objective and the spatial distribution of stimulated fluorescence that occurs from sub-wavelength dipole fluorescent emitters placed throughout the focal volume along the axial direction. Here the induced signal field Eₛᵢ generated at point r near focus is detected at a far field point R where it is mixed with a local oscillator field which is phase coherent with the induced field. In the forward direction the local oscillator field is Eₚᵣ, while in the backscatter direction the local oscillator field is the index gradient backscatter field E_{bs} as shown in FIG. 9. (FIG. 9 shows the backscattered direction of the SISFE dipole emissions as they interfere with an index of refraction gradient induced backscatter). A spatial phase shift for the measured field at a detection point R relative to the phase at the excitation point r can occur, which depends on the excitation and detection geometry. For forward scatter it is assumed that Φ is the spatial phase of the induced field at R relative to the phase at the origination point r, and α measures a similar spatial phase shift between r and R for the excitation field. The forward scattered heterodyne probe signal Sₕₑₜ(R) is shown in SISFE system is shown in Eq. (6).

$${S}_{het} ( R ) = 2 \cdot \frac{n ( {ω}_{pr} ) c}{8 π} \cdot Re \{ {P}_{pr} ( R ) \cdot {E}_{pr}^{*} ( R ) + {P}_{d} ( R ) \cdot {E}_{pr}^{*} ( R ) \} \qquad ( 6 )$$

Where n(ωₚᵣ) is the refractive index of the material at frequency ωₚᵣ, and c is the speed of light. As can be seen in Eq. (6), the heterodyne term consists of the normal SFE gain term and a local oscillator probe and donut field interference term.

However, in the backscatter direction the interference terms in Eq. (6) can be modified. In backscattered detection the excitation probe field generates the induced field at focus. However, in the detection plane the induced field interferes with the index of refraction gradient induced backscattered field E_{bs}, as shown in FIG. 9. E_{bs} depends on Eₚᵣ and the local refractive index change at focus which varies throughout the sample. E_{bs} in tissues is orders of magnitude less intense than the incident probe field.

Thus Eq. (6) can be modified to include interference with E_{bs} as shown in Eq. (7). The backscattered SISFE interference signal is called Bₕₑₜ (R).

$${B}_{het} ( R ) = 2 \cdot \frac{n ( {ω}_{pr} ) c}{8 π} \cdot Re \{ {P}_{pr} ( R ) \cdot {E}_{bs}^{*} ( R ) + {P}_{d} ( R ) \cdot {E}_{bs}^{*} ( R ) \} \qquad ( 7 )$$

E_{bs} can originate from anywhere within the focal spot of the probe beam which can be 2-3 times larger than the SISFE stimulated emission spot. In addition, the amplitude and phase of E_{bs} (r) varies from pixel to pixel and the field can go to zero, and the position of emitters, cannot be correlated with the index of refraction gradients within tissues. In order to isolate the intensity of the stimulated emission signal, the position and intensity of the reference backscatter can be determined by an independent measurement. This can be accomplished interferometrically by measurement of the reference backscatter when the pump beam is modulated off.

The smaller intensity and variability of E_{bs} means that it is possible for Pₚᵣ to be of comparable or greater magnitude than E_{bs}. Thus the measured signal, Iₘ, (Eq. (8)) contains three terms whose magnitudes vary from pixel to pixel. In the absence of I_{bs} the darkfield stimulated emission Iᵢ will be measured.

Iₘ=I_{bs}+Iᵢ+Bₕₑₜ (8)

The forward scattered heterodyne gain signal from a high NA microscope focused on a dipole scatterer, without the donut beam is shown in Eq. (9).

S_{SRG}(R)∝Iₚᵤ(r)Iₚᵣ(r)[−Re{χₚᵣ,r)} cos α(r)+Im{χ(ωₚᵣ,r)} sin α(r)] (9)

The change in the Gouy phase shift at focus is π/2. When a single dipole is present at focus, the induced field exhibits a phase that is spatially invariant, i.e., Φ=0, as opposed to Φ=−π/2 for the emission of a plane of scatterers that is measured in the detector plane. The measured signal in the far field depends on the position-dependent excitation field phase α(r); which as can be seen in Eq. (9) results in different components of the material response being measured depending on the position of the dipole in the focused excitation field along the optic axis. When the dipole particle is placed exactly in the focal plane, then α(z=0)=½π and the gain signal is S_{SRG} Iₘ{χ(ωₚᵣ)}. However, when the particle is placed above or below the focal plane, α≠½π signal contains contributions of Rₑ {χ(ωₚᵣ)}.

Eq. (10) describes the forward scattered SISFE heterodyne signal.

Sₙₑₜ(r)∝[[Iₚᵤ(r)·Iₚᵣ(r)+Iₚᵤ(r)·(Iₚᵣ(r))^{1/2}·(I_{d}(r))^{1/2}·cos Φ]·[−Re{χ(ωₚᵣ,r)} cos α(r)+Im{χ(ωₚᵣ,r)} sin α(r)]] (10)

The cos Φ term represents the Stokes component interference. It is negative when Φ˜π±π/2. Off the optic axis the interference term will rise because of the increasing donut field, and decreasing probe field. Negative gain results from phase change in the molecular polarization enabling absorption rather than emission, and energy transfer from the probe to the donut beam.

In the backscattered direction the heterodyne gain relationship of Eq. (10) can be modified to take into account that the induced field interferes with the index gradient dependent E_{bs}, rather than Eₚᵣ. Unlike the pixel independent reference oscillator probe phase in the forward scattered direction, the position of origination of E_{bs} is variable. In addition, the phase of E_{bs} can vary from pixel to pixel. E_{bs} is substantially weaker than Eₚᵣ, and assuming Mie scatter, the phase change on backscatter can be up to π radians relative to Eₚᵣ(r), depending on the size and structure of the index gradient. This is accounted for by introduction of a new phase term, γ(r), defining the positional separation of both Eₚᵣ(r) and E_{bs}(r) fields as shown in Eq. (8). In the case of confocal imaging E_{br}(r) originates from a sub-wavelength aperture or scatterer and thus undergoes a −π/2 phase shift similar to the Gouy phase through focus.

$${B}_{het} ( R ) ∝ [ {[ {I}_{pu} ( r ) \cdot {I}_{pr} ( r ) )}^{\frac{1}{2}} \cdot {( {I}_{bs} ( r ) )}^{\frac{1}{2}} + {I}_{pu} ( r ) \cdot {( {I}_{bs} ( r ) )}^{\frac{1}{2}} \cdot {( {I}_{d} ( r ) )}^{\frac{1}{2}} \cdot cos ( Φ ) ] \cdot [ - Re \{ χ ( {ω}_{pr} , r ) \} cos γ ( r ) + Im ( χ ( {ω}_{pr} , r ) \} sin γ ( r ) ] ] \qquad ( 11 )$$

### The SISFE PROCESS-QUANTIFICATION

The computation of the Point Spread Function (PSF) provides a quantitative example of the operation of the SISFE system. Here it is assumed that the interfering reference field is a constant and of significant magnitude so that the calculations apply to the forward and backscattered directions. To illustrate the performance of the system it is assumed that both the reference and induced fields originate from the focal plane.

For illustration we use the pulse scenario shown in FIG. 5c and FIG. 6c. In the example, a pump pulsewidth is 0.3 ps and a wavelength band centered at 340 nm is used.

The Stokes components have been chosen to have pulse lengths of 0.3 picoseconds half-width. The probe and donut pulses have central wavelengths of 464 nm (λₚᵣ) and 469 nm (λ_{d}) respectively as illustrated in FIG. 5c. The Stokes pulses are chosen to be π radians out of phase at their temporal center, t=0, which is delayed from temporal center of the pump pulse. The pump and probe beam will probe a NADH electronic transition centered at 465 nm.

As shown in FIG. 1, the focused pump and probe beams are modeled as having Gaussian shape functions, which are the same form as in a confocal microscope as shown in Eq. (12):

I_{pu,pr}(x)=Iₘₐₓ·exp(−4 ln 2(x)²)/d_{co}² (12)

In Eq. (10) the transverse ½ width d_{cot}=0.61λ/NA, λ is the pump or probe beam wavelength and Iₘₐₓ represents either the maximum intensity of the probe beam I_{pr.max} or the maximum intensity of the pump beam I_{pu.max}. In the axial direction d_{cot} is replaced by the axial halfwidth d_{com}=0.88λ/(n−(NA²−n²)^{0.5}).

The donut shape function S_{d} (x) intensity distribution is modeled as shown in Eq. (13).

$${I}_{d} ( x ) = {I}_{d . max} \cdot {sin}^{2} ( \frac{π x}{2 {d}_{d}} ) \qquad ( 13 )$$

I_{d.max} is the maximum annular intensity at the position d_{d} of the annular region, either in the transverse or axial directions. In the axial direction d_{d} increases can be 2.5 times longer than the transverse donut length.

The SISFE system instantaneous probe backscattered gain function in the transverse plane is iPSFₜᵥ (x,t). The entire backscattered system response, PSFₜᵥ (x), is calculated by summation of the instantaneous probe gain iPSFₜᵥ (x,t) in 10 fₛ time steps as shown in Eq. (14), where N is a normalization factor and T is the edge of the temporal backscatter window. In this paper, for simplification, T taken as time point at which the gain is 20% of the peak gain.

PSFₜᵥ(x)=Σ_{t=pump(-T)}^{pump(+T)}iPSFₜᵥ(x,t)/N (14)

The instantaneous axial point spread function is defined as iPSFₐₓ (t,x). In simulations of system performance in the plane of best focus, a square top NADH emitter can be used. The signal in the detector plane is the square of the summed field of scattering points in the focal plane on a 1 nm grid. Focal positions of negative gain subtract from the integrated gain at detector plane.

FIG. 6a-c are graphical depictions of SISFE Point Spread Functions in the axial direction. FIG. 6a is a probe beam axial gain Point Spread Function, iPSFₐₓ at t=0 for systems with peak donut/probe intensity ratio M=0, 1, 2, 4, 6 and 8. Negative gain is shown for large M due to the high intensity of the donut beam. The peak-zero axial 20% width of the instantaneous probe beam gain Point Spread Functions, iPSFₐₓ, throughout the pump pulse is plotted in FIG. 6b. Temporal curves of systems with M=1, 2, 4, 6 and 8 are shown. Above 380 nm dipole backscatter becomes significantly reduced.

The axial direction is important in focal spot size reduction. iPSFₐₓ plots for peak Stokes intensity ratio I_{max.d}/I_{max.pr}, M=0, 1, 2, 4, 6 and 8 are shown in FIG. 6 (a). As shown in the Fig., the iPSFₐₓ for M greater than 4 have negative gain on the spatial edges. This is directly related to the negative gain terms in Eq. (7) and Eq. (8) as the intensity of donut beam greatly exceeds that of the probe beam.

FIG. 6b is the relationship of the dipole emission region to the Stokes beat frequency is shown. It is assumed that backscatter is not efficiently produced for a 20% width of greater than 380 nm. M=3,4,5, and 6.

As t moves away from 0, Δ(t) deviates from π radians, resulting in a broadening of the width of the instantaneous iPSFₐₓ. This is illustrated in the traces in FIG. 6 (b). Plotted are the 20% widths (peak to 20% gain), of iPSFₐₓ at time slices relative to the temporal peak of the pump pulse M=1, 2, 4, 6 and 8. Simulation results for backscatter can fall off rapidly for 20% cutoff of about 0.8λₚᵣ, or ˜371 nm. Systems with axial M<2 are not useful for backscatter generation. It is noted that the intensity at less than 20% of the peak for M>1 trails off significantly faster than a Gaussian curve. The reduction in the intensity tails helps to provide a more sharply defined focal region to facilitate dipole scattering.

FIG. 6c is a time relationship of the envelopes of the interfering donut and probe Stokes components and the pump beam. The pump wavelength is λₚᵤ=340 nm and has a 0.3 ps halfwidth. Two photon excitation at 680 nm can also be used. The Stokes components in this illustration have equal intensity and are it radians out of phase. This is only true at one radial position at focus and only at the temporal center of the pulse. The Stokes components can be delayed 0.2-2000 picoseconds from the pump pulse to enable kasha delay from a higher electronic excited state to the lowest state for fluorescent emission. The Stokes components can be delayed further if the goal is to measure the ratio of fluorescent to non-fluorescent concentrations of fluorescently quenched and unquenched molecules of the same species.

FIG. 6 (c) shows the axial iPSFₐₓ, with a 20% cutoff=371 nm relative to the Stokes beat timing. Through the course of the Stokes pulses the backscatter blinks on and off. The backscatter is on for less than 50% of the time. The closer the Stokes components are in central wavelength the longer the backscatter window relative to the peak of the pump pulse.

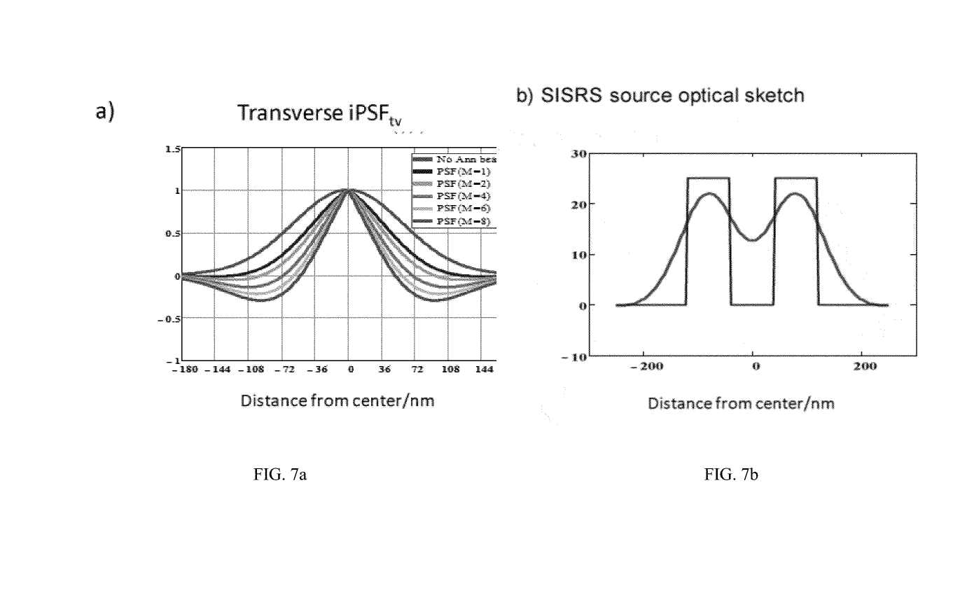

FIG. 7a-b are graphical depictions of backscatter transverse focal spot size, switched performance and system. FIG. 7a is instantaneous iPSFₐₓ, M=3-6. Backscatter 20% cutoff=371 nm. Image resolution is determined by the transverse PSFₜᵥ. FIG. 7 (a) plots the transverse iPSFₜᵥ for M=0, 1, 2, 4, 6 and 8 for the example system. The resolution of the confocal system is much better in the transverse direction so that diameter reduction for dipole scattering is not great. This is fortunate because the π phase plate produces a transverse donut distribution of significantly less intensity than in the axial direction.

FIG. 7b is simulated M=1 scanned signal 2, 80 nm fluorescent scatterers with 350 nm center to center spacing. To illustrate the transverse hyper resolution in backscatter switched SISFE FIG. 7b shows a simulated transverse scan for M=1. The two 80 nm NADH scatterers with a 160 nm period are well resolved. In this simulation the PSFₜᵥ is integrated over the backscatter time period determined by the iPSFₐₓ.

### SIGNAL TO NOISE, PHASE AND SPECTRAL ISOLATION CONSIDERATIONS

SFE imaging is a bright field technique where the stimulated emission forward scatter gain signal is less than 10⁻³ of the probe beam, depending on the number of chromophores in the focused beam. For backscatter SISFE imaging the smaller pixel volume, and use of only a portion of the pump and Stokes pulse widths for imaging, results in lower efficiency in generation of the stimulated Raman scattered field. Fortunately, in the backscatter direction the induced stimulated scattering interferes with the weak gradient index tissue backscattering. In cells, the refractive index change between the cytoplasm and nucleus can be 0.05%, which results in a local backscatter of about 3.4×10-4 of the incident beam. Therefore, the laser power used for a shot noise limited signal in the backscatter direction can be comparable to standard forward scattered SFE imaging. Multiple scattered probe beam photons can be a main source of system noise. Confocal pinholes can be used to reduce collection of multiple scattered probe photons.

The pump and Stokes pulses can be generated by separate synchronized lasers as shown in FIG. 1. The Stokes components can be generated from a single pulse laser and spectrally dispersed and phase modulated. Errors in the relative phase of the Stokes pulses at focus can be generated by thermal, vibrational or humidity induced pathlength errors. Additionally, errors can come from the optical components in the probe and pump paths e.g. the pulse shaper 18; the π waveplate used to create the donut beam; and Gouy phase errors that can be caused by differences in the rate of change of the Gouy phase though focus for the Gaussian probe and donut beams. These random or systematic phase variations can cause the Stokes beams phase difference at the temporal center of the pump pulse to deviate from π radians. However, phase errors are not that damaging because of the Stokes phase difference dependent backscatter gating. Phase errors shift the region of π phase difference of the Stokes pulses relative to the pump pulse center, which can cause a reduction in scattering efficiency.

In the example provided here, the Stokes wavelength difference is 5.0 nm, as shown in FIG. 5c. Smaller Stokes wavelength separation results in longer intervals of gated backscatter, which increase system efficiency. Generation of the Stokes pulses, and isolation of the probe from the donut in signal detection, limit the closeness of the Stokes central wavelengths and shortness of these pulses. A high resolution virtually-imaged phased array (VIPA) disperser can be used to provide high spectral separation and contrast. VIPA's have been shown to resolve the mode structure of a frequency comb, with a 3 GHz mode spacing, from a frequency-stabilized, broadband Ti:Sapphire femtosecond laser, and when used in tandem have demonstrated a contrast of 80 dB.

Some hyper resolution is achieved for diffuse fluorescent molecular distributions. For small scatterers stimulated backscatter will occur throughout the entire SFE focal volume including during Stokes pulses constructive interference. SFE imaging can be used to image the sum of a number of electronic stimulated emissions of multiple molecules, as in imaging of Hemoglobin in capillaries.

A difference of backscattered SISFE from forward scattered SFE is the replacement of the probe local oscillator with the index gradient induced backscatter. The placement of the centroid of the index gradient scatter relative to the stimulated emission site varies from pixel to pixel, and can be measured with better than 100 nm accuracy for the best signal deconvolution.

An alternative method to detect stimulated backscatter is to place a detector around the focal spot to detect multiple scattered photons. Multiple backscattered photons are collected by a photodiode around the aperture of the input microscope. In stimulated fluorescence the absorption and scattering is high and penetration depth in tissue will be less.

SISFE backscattering efficiency from its focal spot can be much less than in forward scattered SFE systems because of the smaller emission volume, and the lower backscatter efficiency. Fortunately, the backscatter local oscillator is also smaller, or not present, enabling the detection of a statistically significant stimulated emission backscatter signal with fewer photons relative to the local oscillator power. With the absence of the requirement for multiple scattering, SISFE is capable of penetrating deeper into tissue and providing better transverse and axial resolution. Clinically, single and multiple backscatter modules can operate in a complimentary fashion.

Further, increasing the intensity of the donut beam reduces the NA at which systems can operate. At M=25, a significant backscatter window is generated at NA=0.85. The negative gain in the axial iPSFₐₓ at M>6 has a variable effect on the field of the backscatter on the detector, depending on the structure of scatterers and local oscillator along the optic axis. For a large scatterer, the Gouy phase through focus introduces a phase angle dependence to the summed field at the detector which scales as the cosine of the variation of the backscatter from the focal position reducing the effect of the edge negative gain on the PSFₐₓ of the image.

### METABOLIC IMAGING WITH SFE AND SISFE

In research, systems can be used, for example, to image tissue cultures, stem cell development, during in-vivo electrophysiological studies for image electrode placement and metabolic correlation with electronic activity. In clinical applications systems can be deployed, for example, in endoscopy, dermatology, intra-surgical definition of structural and metabolically clear margins during removal of malignant tissues, and assessment of tissue viability and drug responsiveness.

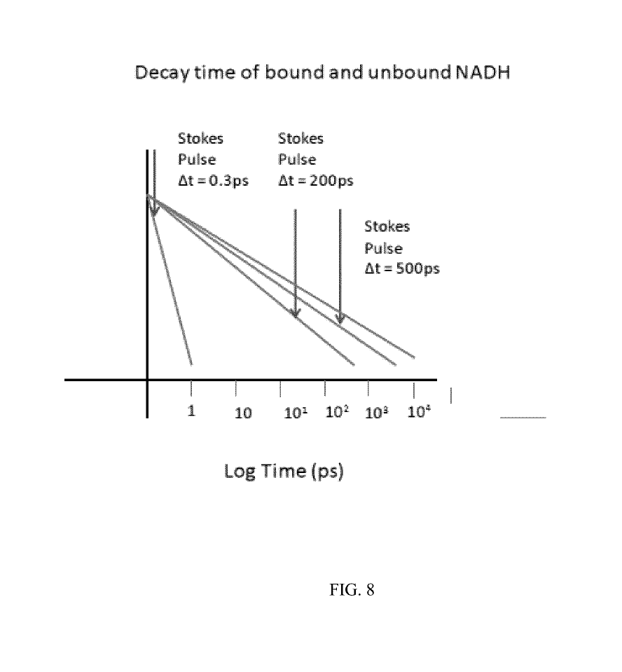

SISFE can be used for metabolic imaging of cells and tissues in order to create an energetic picture of normal, diseased and developing tissues. Imaging of the relative amounts of the enzyme cofactors NADH and FAD and the microenvironment of these metabolic electron carriers can be used to noninvasively monitor changes in metabolism, which is one of the hallmarks of carcinogenesis. Also NADH and FAD can be used to assess the state of developing tissues. When bound to metabolic enzymes, NADH fluorescence quantum yield increases, while FAD quantum yield decreases, which causes variation in the measured fluorescence intensities. SISFE techniques can measure both bound and unbound cofactor concentration and spatially resolve both molecular states. This can be accomplished by changing the delay between the pump pulse and the Stokes pulses to measure the fluorescent lifetimes of bound and unbound states of a particular chromophore as shown in FIG. 8. The hyper resolution of SISFE states can be used to map the distributions of mitochondria in cells in three dimensions to further characterize the metabolic state of cells and tissues.

In another implementation, the use of 2, 3, or 4 photon stimulated emission coupled to 2, 3 or 4 photon stimulated emission for deep tissue imaging without the use of the donut beam is contemplated.

For example, a method and system that uses 2 or more multi-photon excitation and 2 or more multi-photon stimulated emission can be used to cause stimulated fluorescent emission that is significantly red shifted compared to the standard blue or UV fluorescent emission. Red shifting of the emission enables deeper imaging in tissues, by reduction of scattering and absorption. This is called Multi-Photon Stimulated Emission (MP-STEM) imaging. The uses of ≧2 pump photons and ≧2 probe photons can reduce the focal spot size enough to enable direct dipole-like backscatter emission in high numerical aperture systems.

In another example, the use of 2, 3, or 4 photon stimulated emission coupled to 2, 3 or 4 photon stimulated emission can be used to measure fluorescent life by stimulated fluorescence techniques by changing the delay between the pump and probe beams is disclosed. With two or more different temporal delays between the pump and probe beams, molecular fluorescence lifetime can be calculated. The more time delay samples, the more components of lifetime can be measured. This is called stimulated emission Fluorescence Lifetime Microscopy (seFLIM).

Further, one or both of the above techniques can be used to measure the metabolic state of cells deep within tissues via the measurement of the concentration of the metabolic cofactors NADH and NADPH, in both free and bound states and one or both of the above techniques can also be used to image the red shifted UV stimulated emission from proteins and nucleic acids in vivo to image cells without the use of stains.

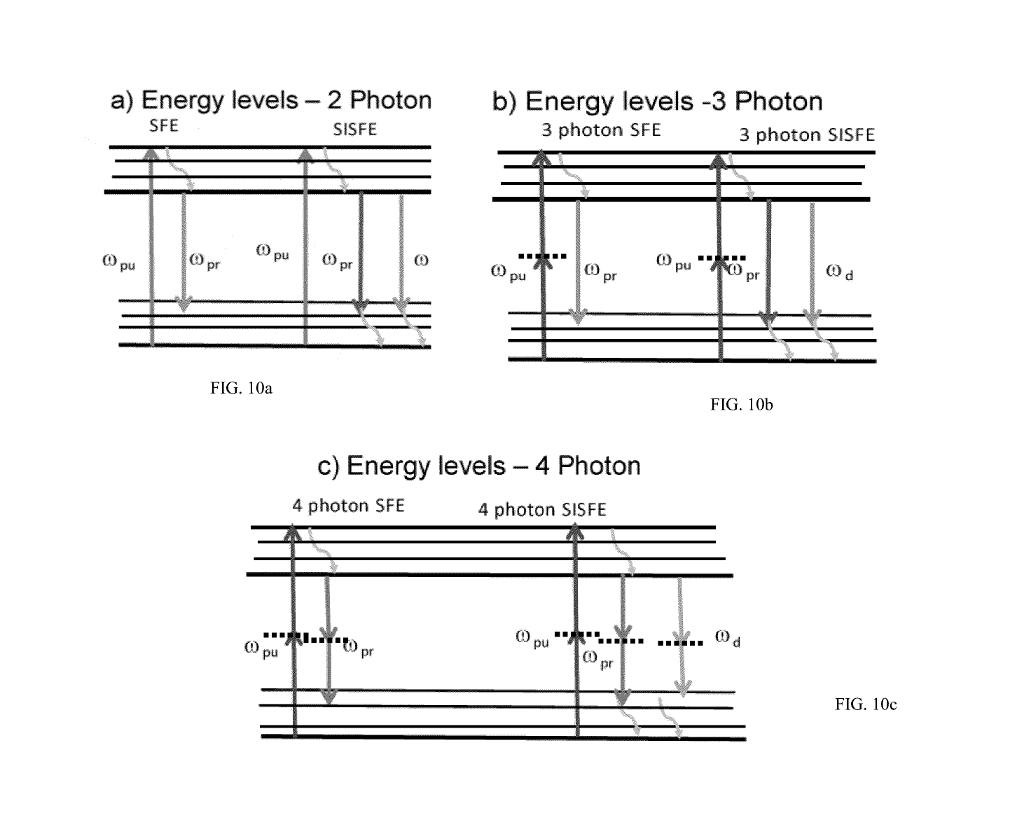

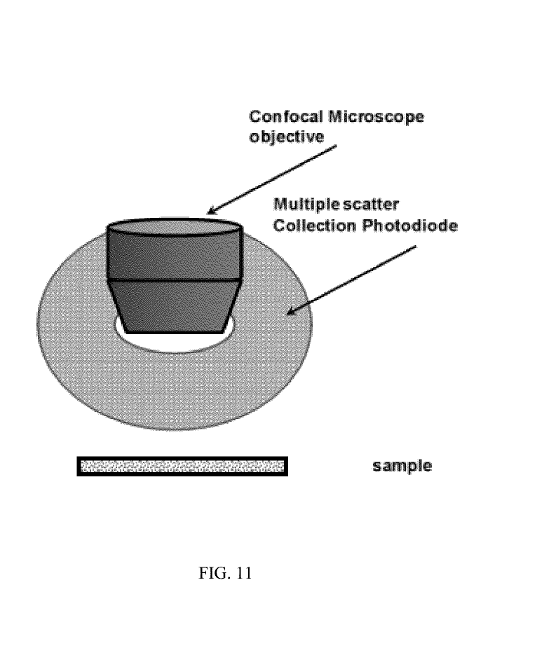

In another implementation, the energetics of 4-photon stimulated emission are shown in FIG. 10c. In this implementation, the system uses 2 photons to excite a real excited state level through a virtual excited state. Then stimulated emission photon beam with photons of ½ the energy difference of the lowest level excited state and an excited level in the ground state manifold are used to stimulate 2 photons added to the stimulated emission beam. This emission occurs as the excited state electron is removed from the excited state to the ground state manifold through a virtual energy level via a two photon stimulate emission process into the ground state manifold. The 4 photon process enables enhanced depth of penetration of imaging of metabolic metabolites and direct imaging of DNA, RNA and protein fluorescence in living tissue. With standard SFE 4-photon imaging the multiple backscattered photons can be collected around the imaging aperture by a photon diode as shown FIG. 11. FIG. 11 shows the microscope objective (32 in FIG. 1) as it collects light for single scatter Dipole backscattered SISFE in 2 photon processes. The backscattered photon diode collects light in multiple backscattered SFE 4 photon processes. For SISFE 4-photon imaging single backscattered light can be collected inside the imaging aperture shown in FIG. 11.

For example, to further enhance the depth of penetration of SFE and SISFE the concept of 4-photon Stimulated Emission is disclosed. In this case two photons are used to create the excited state and two photons are used to create stimulated emission. This 4-photon process is distinct from all previous types of multi-photon microscopy. The two photon stimulated emission adds two photons to the beam used to measure gain. The gain beam is red shifted to the twice the wavelength of the electronic transition that is probed by the emitted light. The 4-photon excitation process disclosed has an excitation cross-section that is the product of a two photon excitation and stimulated emission processes. There are 2 virtual energy transition states in the excitation and emission scheme. However, there is an intermediate low real excited state energy level that accumulates electrons prior to the 2-photon stimulated emission. In addition, with each stimulated emission event two photons are added to the excitation beam and two photons are removed from the stimulated emission beam. This enhances the measured signal. The use of the 4-photon stimulated emission process enables for the first time in-vivo imaging of DNA, RNA and protein UV fluorescent imaging. With standard SFE 4-photon imaging, the multiple backscattered photons can be collected around the imaging aperture. For SISFE 4-photon imaging single backscattered photons can be collected inside the imaging aperture.

While this specification contains many specific implementation details, these should not be construed as limitations on the scope of the disclosed technology or of what can be claimed, but rather as descriptions of features specific to particular implementations of the disclosed technology. Certain features that are described in this specification in the context of separate implementations can also be implemented in combination in a single implementation. Conversely, various features that are described in the context of a single implementation can also be implemented in multiple implementations separately or in any suitable subcombination. Moreover, although features can be described above as acting in certain combinations and even initially claimed as such, one or more features from a claimed combination can in some cases be excised from the combination, and the claimed combination can be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. Moreover, the separation of various system components in the implementations described above should not be understood as requiring such separation in all implementations.

The foregoing Detailed Description is to be understood as being in every respect illustrative, but not restrictive, and the scope of the disclosed technology disclosed herein is not to be determined from the Detailed Description, but rather from the claims as interpreted according to the full breadth permitted by the patent laws. It is to be understood that the implementations shown and described herein are only illustrative of the principles of the disclosed technology and that various modifications can be implemented without departing from the scope and spirit of the disclosed technology.

1. A microscopy system comprising:
a first laser emitting a first laser pulse, the first laser pulse being a pump beam;
a second laser emitting a second laser pulse, the second laser pulse being spectrally isolated for generating a probe beam and a donut beam;
an optical device for combining the pump beam, the probe beam and the donut beam into a combined laser pulse, the probe beam and donut beam having a phase difference that causes a reduction of a focal volume of the combined laser pulse;
a galvanometer scanning system for delivering the combined laser pulse to a focal spot in a focal plane, wherein the reduction of the focal volume of the combined laser pulse initiates a stimulated emission of a targeted molecule, the stimulated emission having dipole-like backscatter; and
a sensor for sensing the dipole-like backscatter.

2. The microscopy system of claim 1 wherein the first laser pulse has a Gaussian beam profile and a sub-picosecond duration.

3. The microscopy system of claim 1 further comprising:
an acousto-optic modulator for modulating the pump beam on and off.

4. The microscopy system of claim 3 wherein the sensor generates an imaging signal corresponding to a gain in intensity of the probe beam computed as the difference between the combined laser pulse with the pump beam on and the combined laser pulse with the pump beam off.

5. The microscopy system of claim 1 further comprising:
a Virtual Imaging Phase Array (VIPA) for spectrally isolating the probe beam and the donut beam from the second laser pulse.

6. The microscopy system of claim 1 further comprising:
a π phase plate for forming the donut beam.

7. The microscopy system of claim 6 wherein the probe beam and the donut beam are sub-picosecond laser pulses of a Stokes module, the probe beam and the donut beam are shifted from a wavelength of the pump laser and directly stimulate emission into a ground state electronic manifold.

8. The microscopy system of claim 7 further comprising:
an optical delay for adjusting pathlengths of the probe beam and the donut beam.

9. The microscopy system of claim 1 wherein the combined laser pulses are delivered in a diffraction limited spot in a focal plane of a high numerical aperture (NA) microscope objective.

10. The microscopy system of claim 1 wherein the combined laser pulses are used to excite an electron into an electronic excited state that emit stimulated emission from its lowest energy excited state level.

11. The microscopy system of claim 1 wherein the galvanometer scanning system moves the focal spot in an X,Y plane.

12. The microscopy system of claim 1 wherein the probe beam and the donut beam are emitted so as to arrive at the focal spot after the pump beam.

13. The microscopy system of claim 1 wherein the probe beam and the donut beam initiate stimulated emission from an excited state of the targeted molecule.

14. A method comprising the steps of:
emitting a first laser pulse, the first laser pulse being a pump beam;
emitting a second laser pulse, the second laser pulse being spectrally isolated for generating a probe beam and a donut beam;
combining the pump beam, the probe beam and the donut beam into a combined laser pulse, the probe beam and donut beam having a phase difference that causes a reduction of a focal volume of the combined laser pulse;
delivering the combined laser pulse to a focal spot in a focal plane, wherein the reduction of the focal volume of the combined laser pulse initiates a stimulated emission of a targeted molecule, the stimulated emission having dipole-like backscatter; and
enabling imaging of the dipole-like backscatter.

15. The method of claim 14 wherein the first laser pulse has a Gaussian beam profile and a sub-picosecond duration.

16. The method of claim 14 further comprising the step of:
modulating the pump beam on and off.

17. The method of claim 16 wherein the sensor generates an imaging signal corresponding to a gain in intensity of the probe beam computed as the difference between the combined laser pulse with the pump beam on and the combined laser pulse with the pump beam off.

18. The method of claim 14 further comprising the step of:
spectrally isolating the probe beam and the donut beam from the second laser pulse.

19. The method of claim 14 further comprising:
forming the donut beam using a π phase plate.

20. The method of claim 19 wherein the probe beam and the donut beam are sub-picosecond laser pulses of a Stokes module, the probe beam and the donut beam are shifted from a wavelength of the pump laser and directly stimulate emission into a ground state electronic manifold.

21. The method system of claim 20 further comprising:
adjusting pathlengths of the probe beam and the donut beam.

22. The method of claim 14 wherein the combined laser pulses are delivered in a diffraction limited spot in a focal plane of a high numerical aperture (NA) microscope objective.

23. The method of claim 14 wherein the combined laser pulses are used to excite an electron into an electronic excited state that emit stimulated emission from its lowest energy excited state level.

24. The method of claim 14 wherein the galvanometer scanning system moves the focal spot in an X,Y plane.

25. The method of claim 14 wherein the probe beam and the donut beam are emitted so as to arrive at the focal spot after the pump beam.

26. The method of claim 14 wherein the probe beam and the donut beam initiate stimulated emission from an excited state of the targeted molecule.
